(19) United States
(12) Patent Application Publication
(10) Pub. No.: US 2004/0152100 A1
(43) Pub. Date: Aug. 5, 2004

(54) Method for sorting cells

(76) Inventor: Katrin Zwiglmaier, Freising (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/479,277

(22) PCT Filed: May 29, 2002
(86) PCT No.: PCT/EP02/05940

(30) Foreign Application Priority Data
May 31, 2001 (DE) 101 26 630.8

(51) Int. Cl. C12Q 1/68

(52) U.S. Cl. 435/6

(57) ABSTRACT

The present invention relates to methods for sorting cell mixtures, with use being made of nucleotide-oriented probes which enable sought-after organisms in a population to be specifically labeled and isolated.

### DESCRIPTION

The present invention relates to methods for sorting cell mixtures, with use being made of polynucleotide probes which enable sought-after organisms in a population to be specifically labeled and isolated.

The identification and characterization of bacteria plays an especially important role in microbial ecology. Since only about 1% of all the bacteria which are present in ecosystems, such as the soil, can be cultured in vitro, culture-independent methods for investigating microorganisms in a very wide variety of ecosystems become increasingly important. However, a variety of population-associated factors have thus far frequently made it more difficult to isolate unknown, noncultivatable organisms. Thus, a population can, for example, be dominated by already known organisms, thereby hindering, and possibly preventing, discovery of the sought-after, previously unknown organisms.

One of the first methods for sorting cells is based on using an infrared laser to manipulate bacterial cells and viruses (Ashkin, A. and Dziedzic, J. M. (1987), Science 235, 1517-1520). It is possible to use this method to accumulate individual cells for a phylogenetic analysis. However, this method is technically very elaborate. For this reason, using infrared lasers to manipulate cells for the purpose of concentrating noncultivatable organisms is only used in a very few instances.

Flow cytometry (FCM) represents another method for concentrating bacterial cells from a mixture of cells. The method makes it possible to analyze cells rapidly (more than 10³ cells per second) and to sort individual cells in a cell population. In this connection, a variety of FCM criteria, such as cell size, cell morphology, DNA content and specific staining with fluorescence-labeled antibodies (Porter, J., Edwards, C., Morgan, J. A. W. and Pickup, R. (1993), Appl. Environ. Microbiol. 59, 3327-3333) or fluorescence-labeled rRNA-directed olinucleotide probes (Wallner, G., Erhardt, R. and Amann, R. (1995), Appl. Environ. Microbiol. 61: 1859-1866), make it possible to isolate cells in the flow cytometer and thus to sort cell populations derived from a very wide variety of systems. However, flow cytometry is a technically very elaborate method which requires expensive laboratory equipment and is only of low specificity, with this being dependent on the FCM criteria which are investigated in each case.

A method for depleting bacterial cells from cell mixtures which is culture-independent, rapid and flexible uses biotin-labeled rRNA probes (Stoffels, M. et al. (1999), Environmental Microbiology 1(3), 259-271). In this method, the sought-after cells are labeled with biotinylated polyribonucleotide probes by in-situ hybridization and then incubated with paramagnetic streptavidin-coated particles. The target cells which are labeled in this way are then separated off from the remaining cells in the cell mixture through a steel wool-filled column which is located in the field of a permanent magnet. This method makes it possible to use the biotin-labeled rRNA probes to sensitively and specifically label the sought-after cells in the cell mixture. However, a disadvantage of the method is that the concentration of the labeled cells is effected using binding proteins (in this case, biotin-streptavidin binding) which frequently also enter into nonspecific bonds, thereby hindering any highly specific depletion of the sought-after cells.

In summary, the literature has not thus far described any suitable culture-independent method for sorting sought-after cells from a cell mixture, with this method enabling individual target cells to be species-specifically labeled and to be isolated in a highly sensitive and highly specific manner, and also being technically simple, automatable and economical.

The present invention was therefore based on the object of providing methods for sorting a cell mixture containing at least one target cell, which methods do not suffer from the abovementioned disadvantages in accordance with the prior art.

According to the invention, this object is achieved by means of a method for sorting, or for detecting, at least one target cell, which contains at least one sought-after nucleotide sequence, in a cell mixture containing this target cell, where

(1) the cell mixture is treated, under hybridization conditions, with at least one polynucleotide sequenced, as probe, which sequence is complementary to the sought-after nucleotide sequence of the target cell,

(2) the cell mixture which has been treated in this way is contacted, under hybridization conditions, with a polynucleotide sequence which is immobilized on a solid support and which is complementary to the probe polynucleotide sequence, and

(3) the target cells which are immobilized on the solid support are separated off from the cells which are not immobilized.

The use, according to the invention, of polynucleotide sequences as probes, which sequences are selected such that the probe nucleotide sequence is complementary to a sought-after nucleotide sequence of the target cell and complementary to a polynucleotide sequence which is linked to a solid support, provides a cell mixture-sorting method which enables a sought-after target cell to be depleted from the cell mixture in a highly specific and sensitive manner. In this connection, the method according to the invention is based on the principle of the double hybridization of the polynucleotide probe.

The term “complementary”, as used herein, includes the possibility that the nucleotide sequences are completely complementary, i.e. undergo complete base pairing. However, according to the invention, complementary sequences are also to be understood as being sequences in which less than 100%, for example more than 80%, preferably more than 90% and more preferably more than 95%, of the bases are complementary, i.e. are able to undergo base pairing. In addition, it is not necessary, according to the invention, for the complete probe nucleotide sequence to be complementary to the sought-after nucleotide sequence; on the contrary, a complementarity of constituent regions of the sequences, in particular of constituent regions having a length of at least 15, more preferably at least 18, and particularly preferably at least 20, bases is adequate within the meaning of the invention. The important thing is that the probe nucleotide sequence hybridizes, under hybridization conditions, with the sought-after nucleotide sequence of the target cell and with the polynucleotide sequence which is linked to a solid support.

Due to the low technical input of the method according to the invention, it can be used in any conventional standard laboratory for isolating any kind of previously unknown organisms, such as prokaryotic cells and eukaryotic cells, in particular noncultivatable bacterial cells, yeast cells and animal cells, for a microbiological characterization, such as molecular analysis or genomic investigation. In this connection, the method according to the invention provides two possible isolation routes. In the first place, the previously unknown organism, preferably a eukaryotic or prokaryotic cell, can be labeled with a species-specific polynucleotide probe and isolated or concentrated using a solid support. In the second place, organisms which occur frequently in the cell population can be labeled with either appropriate species-specific polynucleotide probes or less specific polynucleotide probes and separated off using a solid support. This results in the dominant cells in the cell population being depleted and the previously unknown organism being concentrated as a result.

Step (1) of the method according to the invention comprises treating a cell mixture to be investigated with at least one polynucleotide probe which is complementary to at least one sought-after nucleotide sequence in the sought-after target cell and is based on in-situ hybridization between the complementary regions of the polynucleotide probe and of the sought-after nucleotide sequence in the target cell in the cell mixture. Suitable hybridization conditions are known to the skilled person and are described, for example, in Maniatis, T., Fritsch, E. F. and Sambrook, J., 1982, Molecular Cloning: A Laboratory Manual, Cold Spring Harbor Laboratory, Cold Spring Harbor, N.Y. and Amann, R., Ludwig, W. and Schleifer, K. H., 1995, Phylogenetic identification and in situ hybridization of individual microbial cells without cultivation, Microbiol. Rev. 59, pp. 143-169. The in-situ hybridization in accordance with the present invention preferably takes place in hybridization buffer for from 5 to 30 hours and at from 50 to 60° C., preferably at from 50 to 55° C., and particularly preferably at 53° C. Where appropriate, the hybridization mixture can be incubated, prior to the in-situ hybridization, for from 15 to 30 minutes, preferably 20 minutes, at from 70 to 85° C., preferably 80° C.

The polynucleotide probes on which the method according to the invention is based have the crucial property that, during a hybridization, they do not all completely penetrate into the target cell. A portion of the polynucleotide probe does not gain entry into the cell and remains outside the cell wall of the target cell. It is assumed that networks are formed from probes which are anchored to the cells by way of some probe molecules which are hybridized intramolecularly with target molecules such as rRNA or DNA. According to the invention, this fraction of the polynucleotide probe network which is located outside the target cell possesses a region which is complementary to a nucleotide sequence which is linked to a solid support. Using this phenomenon, it is possible to label the sought-after target cells species-specifically with the polynucleotide probe in step (1) of the method according to the invention and to use the fraction of the polynucleotide probes which is located extracellularly to separate off these target cells from cells which are not labeled with the polynucleotide probes (step (2) of the method according to the invention).

Step (2) of the method according to the invention comprises linking or immobilizing the cell mixture target cells, which were labeled with a polynucleotide probe in step (1), onto a solid support. According to the present invention, the linking or immobilizing of the polynucleotide probe-labeled target cells is effected by way of a hybridization between the sequence region of the polynucleotide probe which is located extracellularly and a nucleotide sequence which is complementary to the sequence region and which is fixed to, or immobilized on, a solid support. The hybridization is preferably effected using known hybridization methods and under known hybridization conditions (see, e.g., Maniatis, T., Fritsch, E. F. and Sambrook, J., 1982, Molecular Cloning: A Laboratory Manual, Cold Spring Harbor Laboratory, Cold Spring Harbor, N.Y.). The hybridization in accordance with the present invention preferably takes place for from 1 to 2 hours at from 50 to 60° C., preferably at from 50 to 55° C., and particularly preferably at 53° C. The polynucleotide probe-labeled target cells can thereby be immobilized and concentrated on the solid support which is coated with the nucleotide sequence which is complementary to the probe nucleotide sequence.

If the polynucleotide probe-labeled target cells constitute the previously unknown organism, the isolation of the unknown organism is then effected by immobilizing and/or concentrating it on a solid support. If, on the other hand, the dominant organisms in a cell population are polynucleotide probe-labeled, these organisms are then removed or depleted from the cell population by being immobilized on a solid support, leading to the previously unknown organism being concentrated in the cell population medium.

Known support materials, such as microtiter plates, e.g. microtiter plates which possess a special surface coating, and thus enable polynucleotides to be coupled noncovalently, hydrophobically or hydrophilically, and conventional microtiter plates, in which the coupling of the polynucleotide is effected by way of a covalent bond, or by way of amino linkers or phosphorylation; membranes; particulate support materials, which can be coated directly with polynucleotides or in which the binding is effected by way of binding proteins (streptavidin-biotin binding); and biochips, can be used as solid supports in the method according to the invention. Preference is given to using microtiter plates in the method according to the invention since they are suitable for a high sample throughput and enable the method to be automated.

In a preferred embodiment of the invention, a microtiter plate, preferably Maxisorp Micro Wells (NalgenNunc Int., Naperville, Ill., USA), is used as the solid support when depleting dominant target organisms since microtiter plates exhibit very high binding capacity. Moreover, particulate support materials are preferred if the dominant target organisms are eukaryotic cells since the eukaryotic cells, which are substantially larger than prokaryotic cells, are easily washed away when linked to a plane surface such as microtiter plates.

If, on the other hand, the method according to the invention is used, in another embodiment, to extract a sought-after cell type from a large cell mixture, and to isolate it by linking, use is preferably made of particulate support materials which are coated, for example, with DNA. After it has been concentrated, a target organism which is bound to a particulate support can be immediately subjected to further processing and can, in the linked state, be used, for example, for a microscopic analysis, a PCR or a sequencing.

According to the present invention, known polynucleotide coating methods are used to coat the support materials with the polynucleotide sequence which is to be fixed and which is complementary to the polynucleotide probe (for microtiter plates, see, for example, Ezaki, T., Hashimoto, Y. and Yabuuchi, E., 1989, Fluorometric DNA-DNA hybridization in microdilution wells as an alternative to membrane filler hybridization in which radioisotopes are used to determine genetic relatedness among bacterial strains, Int. J. Syst. Bacteriol. 39, pp. 224-229; for membranes and other support materials, see, e.g., Maniatis, T., Fritsch, E. F. and Sambrook, J., 1982, Molecular Cloning: A Laboratory Manual, Cold Spring Harbor Laboratories, Cold Spring Harbor, N.Y.; or in accordance with the manufacturer's instructions. In this connection, the polynucleotide probe is preferably coupled to the solid support covalently, by means of adsorption or by way of specific binding partners. The polynucleotide sequence which is fixed to the support can be any arbitrary nucleic acid sequence and is preferably a DNA sequence or an RNA sequence.

In step (3) of the method according to the invention, the polynucleotide probe-labeled target cells which are linked to the solid support are separated off from unlinked cells in the cell mixture and thereby isolated and concentrated or depleted, with it being possible for the target cell in accordance with the method according to the invention either to be a previously unknown organism or at least one dominant organism.

In the method according to the invention, use is made of novel polynucleotide probes which comprise the following sequence segments:

(i) at least one first polynucleotide sequence which is complementary to a nucleotide sequence in the target cell, with the polynucleotide sequence preferably being complementary to a highly variable nucleotide sequence in the target cell, and

(ii) at least one second polynucleotide sequence which is complementary to a polynucleotide sequence which is fixed to a solid support.

The novel polynucleotide probes of the method according to the invention make it possible to achieve a cell sorting which is species-specific and highly selective. The sequence segments (i) and (ii) of the polynucleotide probe can be selected at will as long as the abovementioned criteria are fulfilled. The total length of the polynucleotide probe is preferably at least 50 nucleotides, preferably at least 100 nucleotides, and more preferably at least 150 nucleotides. While the upper limit of the total length is not restricted, the polynucleotide probes preferably have a length of at most 1000 nucleotides, more preferably at most 800 nucleotides.

According to the invention, the polynucleotide probe can be prepared in one piece, e.g. synthetically, by means of in-vitro transcription or PCR, with the polynucleotide probe template comprising the two sequence segments (i) and (ii), or be assembled from the two sequence segments (i) and (ii) after they have been synthesized.

At least the sequence segment (i) of the polynucleotide probe is preferably complementary to a highly variable sequence within the sought-after target cell. Such highly variable sequences can, for example, be weakly conserved regions of the cellular rRNA, such as highly variable regions of the 23S rRNA or 16S rRNA, or of the 28S rRNA or 18S rRNA, or high-copy, medium-copy and low-copy plasmids. These sequences make it possible to effect a reliable hybridization with the polynucleotide probe which is penetrating into the target cell. However, it has also been possible to carry out the method successfully using probes directed against chromosomal DNA.

In a preferred embodiment, the sequence segment (i) of the polynucleotide probe according to the invention possesses a sequence which is complementary to a highly variable region of the 23S rRNA (domain III) and/or 16S rRNA. In this case, recourse can be had to a data set of at present approx. 2000 or 22 000 sequences when selecting the probe sequence. However, in a special case, the frequently occurring sequence which is relevant for a target cell type, but which has not previously been described, can also be initially identified and then used as the starting point for constructing suitable polynucleotide probes. In the case of an unknown species, it is possible, for example, to use primers which bind to strongly conserved sites, with it not being necessary to know the sequence which lies in between them.

The sequence segment (i) of the polynucleotide probe should preferably have a length of at least 15, preferably at least 18, particularly preferably at least 20, and more preferably at least 25, nucleotides in order to ensure that the binding between the polynucleotide probe and the nucleic acid of the target cell is specific. However, it is frequently sufficient for an in-vitro hybridization if the sequence segment (i) has a length of preferably ≦100 nucleotides, more preferably ≦50 nucleotides and most preferably ≦40 nucleotides.

The sequence segment (ii) of the polynucleotide probe is complementary to a nucleotide sequence which is fixed to a solid support. In a case of a composite polynucleotide probe, this sequence segment is preferably selected such that simple, rapid and quantitative hybridization takes place with the nucleotide sequence which is fixed to the solid support.

In a preferred embodiment of the method according to the invention, the polynucleotide probe is a ribonucleic acid probe (RNA probe). This probe can be prepared in a known manner, e.g. synthetically. The RNA probes of the method according to the invention are preferably prepared by means of traditional in-vitro transcription (see, e.g., Maniatis, T., Fritsch, E. F. and Sambrook, J., 1982, Molecular Cloning: A Laboratory Manual, Cold Spring Harbor Laboratory, Cold Spring Harbor, N.Y.). RNA probes advantageously make it possible to achieve strong and quantitative hybridization between the probe sequence and the target rRNA and form extremely stable RNA-RNA hybrids.

In another embodiment of the method according to the invention, the polynucleotide probe is a deoxyribonucleic acid probe (DNA probe). The DNA probe can be prepared by traditional methods, either synthetically by means of oligonucleotide synthesis or else by means of amplification using PCR (J. Zimmermann et al., Syst. Appl. Microbiol. 24(2) 238-244 (2001)) (see, e.g., Maniatis, T., Fritsch, E. F. and Sambrook, J., 1982, Molecular Cloning: A Laboratory Manual, Cold Spring Harbor Laboratory, Cold Spring Harbor, N.Y.).

In addition, the polynucleotide probe can, in another embodiment of the method according to the invention, be labeled with suitable labeling substances, such as biotin and digoxygenin, or fluorescence dyes, which are used in epifluorescence microscopy, such as fluorescein, Cy3, Cy5, rhodamine and Texas Red, in order to enable the polynucleotide probe-labeled target cells to be subsequently detected. Preference is given, in the present invention, to using biotin and digoxygenin as labeling reagents. This makes it possible to monitor the cell sorting which is performed using the method according to the invention in a microscope by using streptavidin-fluorescein or antidigoxygenin-fluorescein to detect the polynucleotide probe-labeled target cells. It furthermore makes it possible to quantify the target cells which are linked to the solid support, preferably a microtiter plate, either by counting the target cells under a microscope or by using streptavidin-peroxidase or antidegoxygenin-peroxidase to detect them photometrically. However, the invention also provides for the target cells which are linked to the solid support to be quantified by means of semiquantitative PCR. In this case, a PCR is carried out using dilution series of the cell suspension of the cell mixture to be investigated before and after the concentration/depletion. A comparison of the dilution step up to which a PCR product is still formed makes it possible to quantify the target cells which are linked to the solid support.

For working with environmental samples, counting cells in a microscope is no longer practicable for quantifying successful depletion and semiquantitative PCR gives values which are not very precise. In this case, it is expedient to use a confocal laser scanning microscope (LSM), e.g. together with suitable software.

For this, a part of the sample is applied, after the depletion, to a microscope slide and hybridized with at least two oligonucleotide probes which are fluorescence-labeled differently: 1. A probe which binds to all the cells which are present in the sample (Eub338; Daims H. et al., System. Appl. Microbiol. 22:434-444 (1999)), 2. A probe which is specific for the depletion target cells. The LSM is now used to take photographs of at least 10 arbitrarily selected microscope fields of view. The analysis is performed, for example, using commercially available software, such as Quantimet 570 (Leica Cambridge Ltd., Cambridge, UK), NIH Image (Wayne Rasband, NIH, Bethesda, Md., USA) or Artek 810 Image Analyzer (Artek Systems Corp., Farmingdale, N.Y., USA), which is based on comparing the total area of the bacteria with the area of the target cells. By taking photographs before and after the cell sorting, it is thus possible to quantify the depletion.

In another embodiment of the method according to the invention, the cell mixture to be investigated is first of all fixed, preferably using paraformaldehyde, particularly preferably using 4% paraformaldehyde. The fixing takes place for from 10 to 16 hours, preferably for 12 hours, at, e.g., 4° C. While fixing is not absolutely necessary in the case of yeasts and other eukaryotic organisms, it is to be recommended. Bacterial cells are preferably fixed in order to make the cells more readily accessible to the probe.

Because of its broad applicability, its high specificity, its high sensitivity and the possibility of automating it, the method according to the invention can be used in wide areas of microbiology. Thus, virtually all previously unknown, cultivatable and/or noncultivatable cells in any arbitrary cell population can be detected and isolated and are in this way available for subsequent molecular analysis and/or genomic investigation. For example, the method has been successfully applied to some organisms which, according to the Federal Epidemics Act, are rated as belonging to risk group 2: Acinetobacter calcoaceticus ATCC 17978, Enterobacter aerogenes, Escherichia coli ATCC 11775T, Klebsiella pneumoniae DSM 30104 and Pseudomonas aeruginosa DSM 6279. It has been possible to successfully deplete the organisms in artificial mixtures. In addition, investigations have been carried out using environmental samples (sewage sludge from the settling tank belonging to the animal body utilization plant at Kraftisried) with which the organisms have been admixed. In this connection, it was possible to demonstrate successful depletions using Escherichia coli and Pseudomonas aeruginosa. The use, according to the invention, of the polynucleotide probes for sorting a cell mixture circumvents the relatively nonspecific concentration of the target cells which is achieved using binding proteins. Furthermore, the method according to the invention facilitates release of the target cells from the linked solid support.

The following figures and examples are intended to illustrate the invention in more detail.

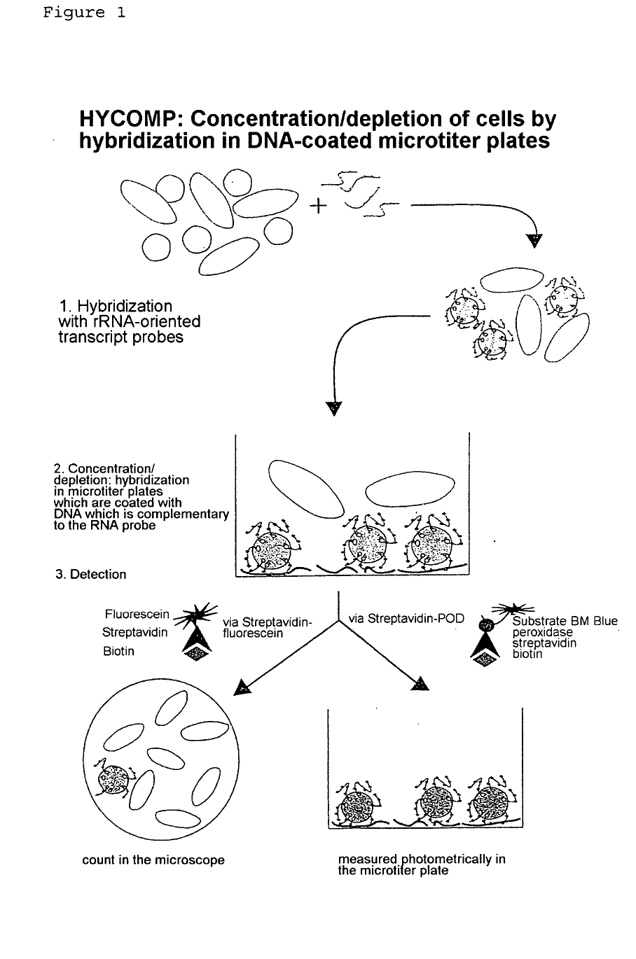

FIG. 1 shows a diagram of the method according to the invention for sorting cell mixtures. The target cells are labeled by performing a hybridization between a biotin-labeled polynucleotide probe according to the invention and a sought-after nucleotide sequence in the target cell. The target cells are immobilized and depleted by means of a subsequent hybridization between the region of the polynucleotide probe which is located extracellularly and a polynucleotide sequence which is fixed to a solid support (preferably a microtiter plate). The immobilized target cells can be detected by the streptavidin-fluorescein detection of the biotin-labeled probe in a microscope or else photometrically by the streptavidin-peroxidase detection of the biotin-labeled probe on the solid support.

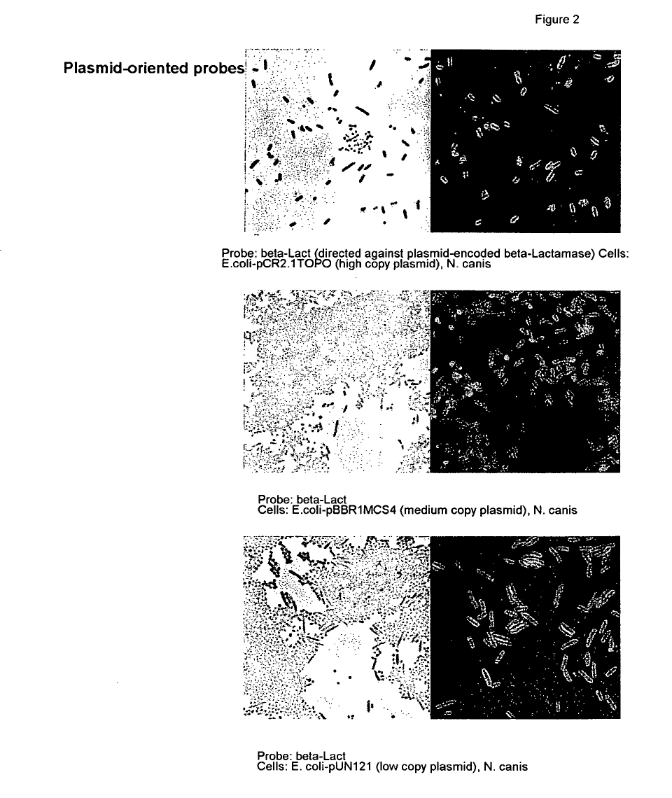

FIGS. 2a, b and c show, in microscope photographs, a recording of the results obtained with the method described in Example 3 when using plasmids as anchoring molecules for the polynucleotide probes. The figures in each case compare: on the left, a phase contrast photograph without probe; on the right, the same photograph with the fluorescent probe, which shows a halo around the target cells. The specificity of the labeling of these target cells can be clearly seen.

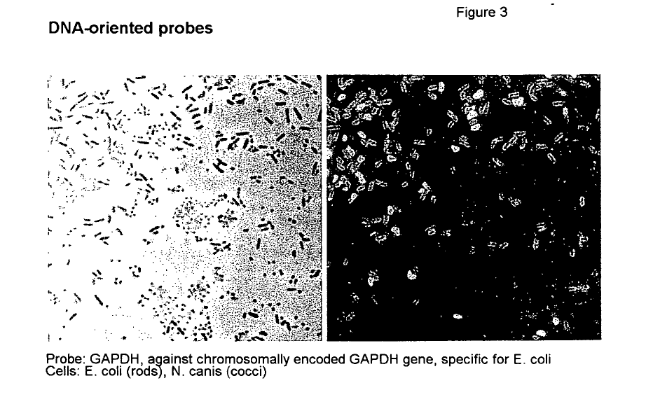

FIG. 3 shows microscope photographs, which are analogous to those in FIG. 2, for Example 4 (depletion using genomic DNA as the anchor for a polynucleotide probe).

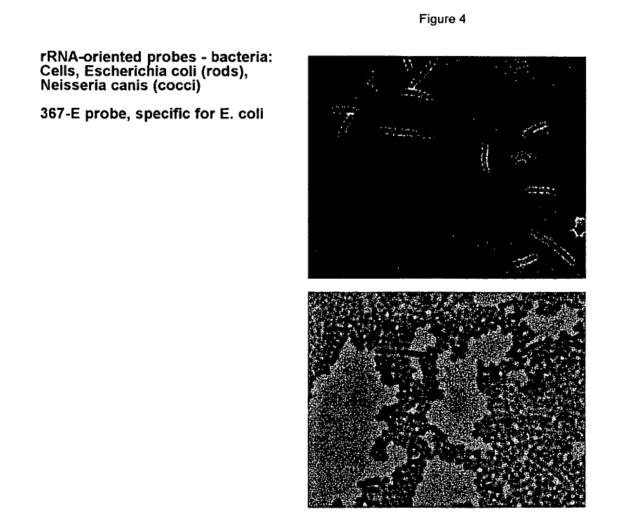

FIG. 4 shows, at the top and in analogy with the manner described in FIG. 2, a phase contrast photograph of E. coli which are labeled with the E. coli-specific probe 367-E and which display typical halos. The same photograph without a probe is given below, with this photograph now showing the Neiseria canis cocci, which are predominantly present, in addition to the E. coli rods.

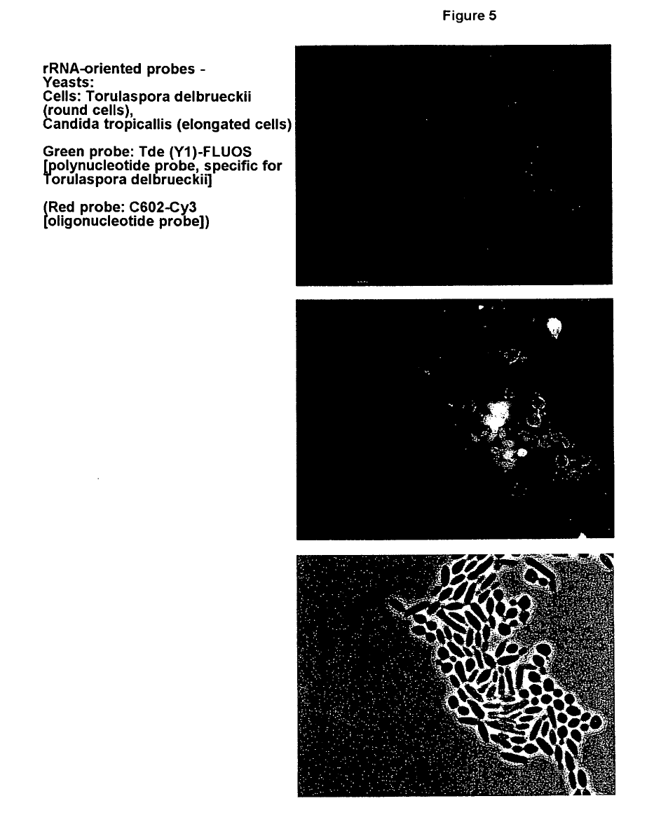

FIG. 5 shows microscope photographs in analogy with FIG. 4. Only the probe specific for Torulspora delbrueckii was used in the upper picture; two probes are used in the middle picture: the specific probe for Torulaspora delbrueckii and the probe which fluoresces red and binds to all the cells, thereby visualizing Candida tropicalis in addition to Torulaspora delbrueckii. The lower photograph corresponds to the upper two photographs but without a probe being added.

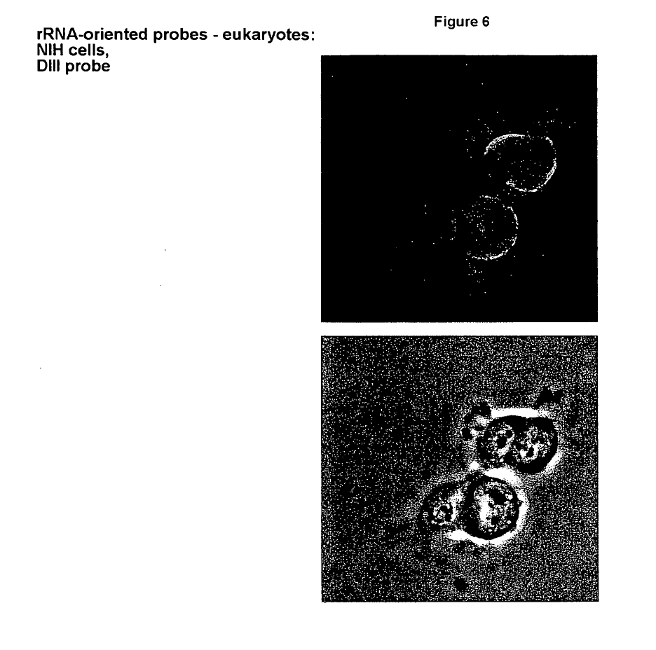

FIG. 6 shows photographs in analogy with FIG. 4. In the upper picture, the eukaryotes are labeled with rRNA probes with the formation of the typical halo. The lower picture shows the same photograph without a probe.

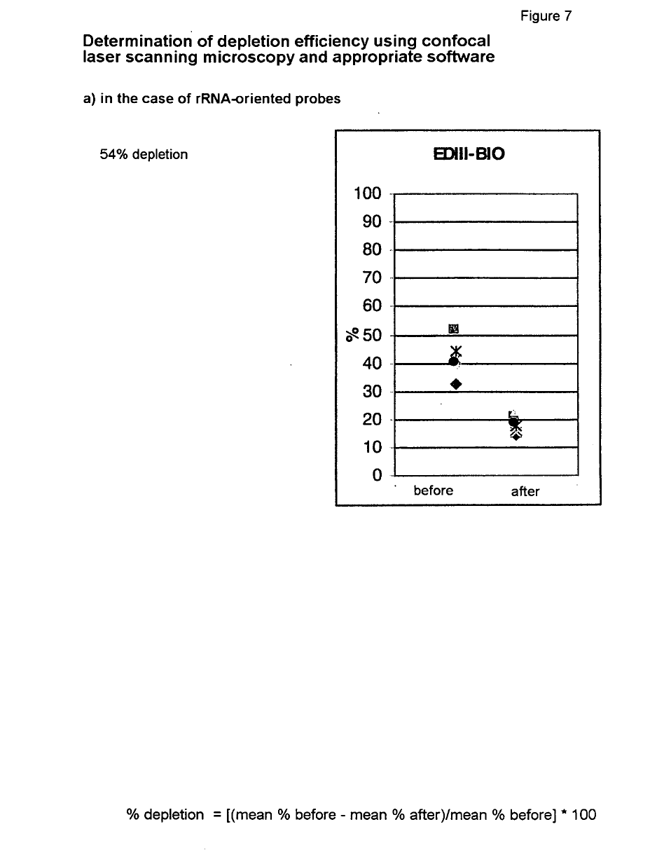

FIG. 7 shows a diagram which depicts the percentage of target cells before and after depletion using an rRNA-oriented probe.

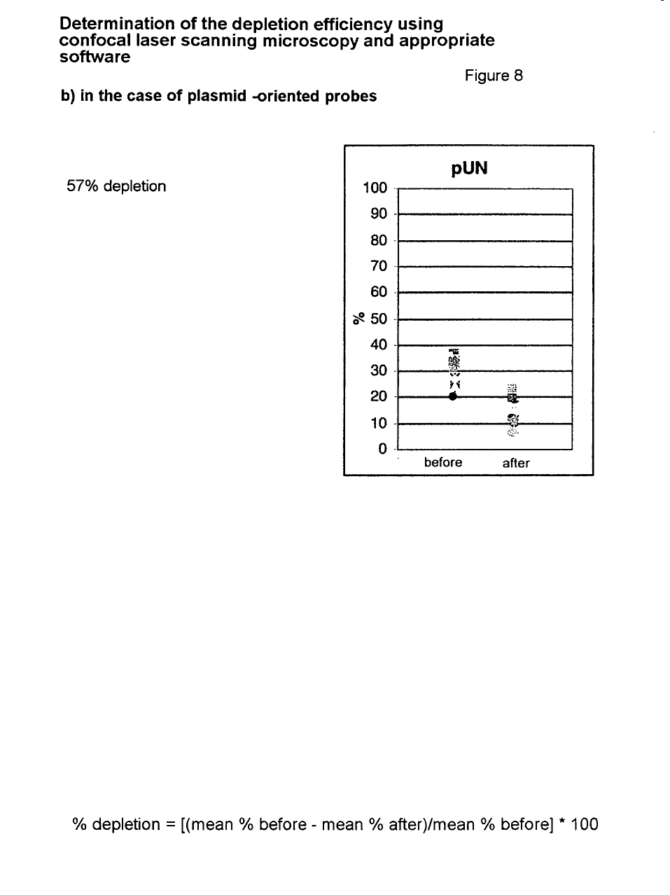

FIG. 8 shows a diagram as in FIG. 7 but for a plasmid-oriented probe.

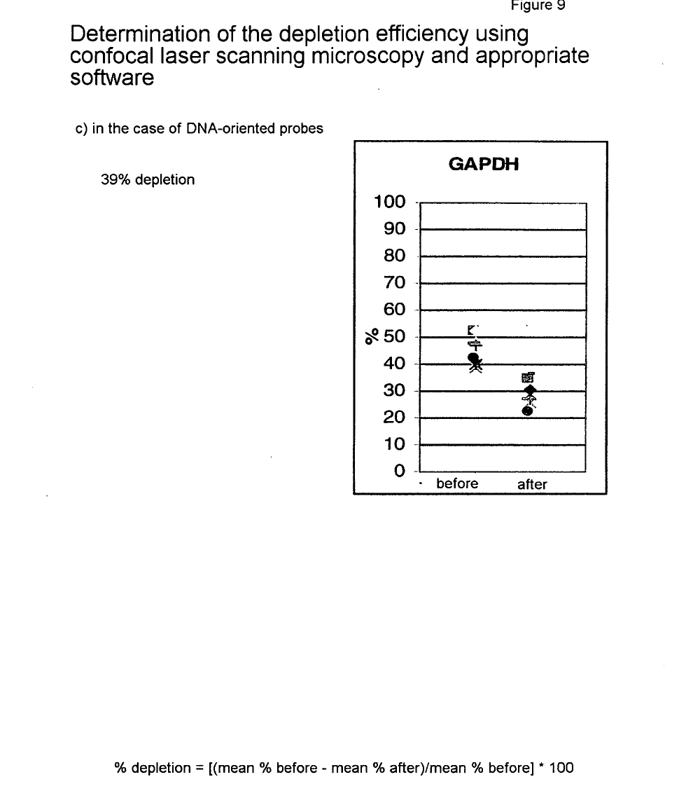

FIG. 9 shows a diagram as in FIG. 7 for a DNA-oriented probe.

### EXAMPLE 1

Using the Method According to the Invention to Sort a Cell Mixture

### EXAMPLE 1a

Cell Fixing

A cell suspension is centrifuged down (15 min, 5000 rpm) and the cell pellet is then resuspended in PBS (137 mM NaCl, 10 mM Na₂HPO₄/KH₂PO₄, 2.7 mM KCl, pH 7.2). 3 vol. of fixing solution (4% paraformaldehyde [w/v] in PBS, pH 7.0) are added to the cell suspension. The fixing takes place at 4° C. for 12 hours. The cells are then centrifuged down (2 min, 12 000 rpm), washed with 1 ml of PBS and finally taken up in 0.5 ml of PBS. 1 vol. of abs. EtOH is added to the cells to enable them to be stored. The cells which have been fixed in this way can be stored for some months at −20° C.

### EXAMPLE 1b

Using In-Vitro Transcription to Prepare rRNA-Oriented RNA Polynucleotide Probes

PCR is used to amplify a highly variable region of the 23S rRNA (domain III) from purified genomic DNA. To do this, use is made of the modified primer pair 1900VN and 317RT3, having the following nucleotide sequences. 1900VN: 5′-MADGCGTAGBCGAWGG-3′, 317RT3: 5′-ATAGGTATTAACCCTCACTAAAG GGACCWGTGTCSGTTTHBGTAC-3′. The primer 317RT3 contains the promoter sequence for the T3 RNA polymerase (underlined) which is required for the in-vitro transcription. The PCR amplification is carried out in accordance with the following protocol: 100 ng of genomic DNA, in each case 50 pmol of 1900VN and 317RT3, 80 nmol of DNTP, 10×PCR buffer (Takara Suzo, Co., Otsu, Japan) and 3U of Taq polymerase (Takara rTaq) are made up with H₂O to a volume of 100 μl. After an initial denaturation of 94° C. for 3 min, there then follow 30 cycles of denaturation at 94° C. for 1 min, primaer annealing at 50° C. for 1 min and primer extension at 72° C. for 1 min, as well as a final elongation at 72° C. for 5 min. The PCR products are purified using a QIAquick matrix (QIAGEN, Hilden, Germany). From 1 to 2 μg of PCR amplificates are used for the in-vitro transcription.

During the transcription, the probes are labeled as desired with biotin or digoxygenin. The composition of the transcription mixture is as follows: 200 nmol of NTPs (consisting of ATP, CTP, GTP, UTP and biotin-16-UTP (Roche) or DIG-11-UTP (Roche) in the ratio 1:1:1:0.35:0.65), 3 μl of 10× transcription buffer (Roche), 3 μl of T3 RNA polymerase (Roche), 1.5 μl of RNase inhibitor (Roche) and from 1 to 2 μg of PCR product in a final volume of 30 μl. The mixture is incubated at 37° C. for from 3 to 4 h. 3 μl of DNaseI (Roche, RNase-free) are then added and the mixture is incubated at 37° C. for a further 15 min. The reaction is stopped by adding 3 μl of 0.2 M EDTA and the RNA is precipitated, at −20° C. for 2 h, with 16 μl of NH₄-acetate and 156 μl of abs. EtOH. The RNA is centrifuged down (15 min, 14 000 rpm, 4° C.), washed with 70% EtOH and finally taken up in 50 μl of H₂O+1 μl of RNase inhibitor and measured photometrically. The probe can be stored at −20° C. for from 2 to 3 months.

### EXAMPLE 1c

Hybridizing with Biotin-Labeled or DIG-Labeled Polynucleotide Probes

2 vol. of abs. EtOH are added to from 5 to 10 μl of PFA-fixed cells in an 0.5 ml reaction tube and the mixture is incubated at room temperature for 3 min. The cells are then centrifuged down (3 min, 12 000 rpm), washed with PBS and finally taken up in 30 ml of hybridization buffer (75 mM NaCl, 20 mM Tris pH 8.0, 0.01% SDS, 80% formamide). From 0.5 to 4 μg of probe are added and the mixture is then incubated at 80° C. for 20 min in order to denature RNA secondary structures. This is then followed by the hybridization, at 53° C. for 5 to 16 h, in a hybridizing oven.

### EXAMPLE 1d

Coating the Microtiter Plates

Use is made of Maxisorp MicroWells (NalgenNunc Int., Naperville, Ill., USA) which are coated with DNA which is complementary to the RNA probe. For this purpose, the sequence of domain III of the 23S rDNA which is used as probe is amplified by PCR using the protocol described under item 1b. The PCR products are purified by precipitating with 0.1 vol. of 5M NaAC, pH 5.5, and 2 vol. of abs. EtOH. 1 μg of PCR product in 50 μl of PBS/MgCl ₂ (137 mM NaCl, 10 mM Na₂HPO₄/KH₂PO₄, 2.7 mM KCl, 100 mM MgCl₂, pH 7.2)+50 μl of H₂O are added per microtiter well. The plates are sealed with adhesive foil (Nunc, Naperville, Ill., USA), denatured at 94° C. for 10 min on a heating block and then incubated at 37° C. for 1 h. The plates are tapped out on paper and dried at 60° C. for from 1 to 2 h in an oven. When sealed with adhesive foil, the coated plates can be kept at room temperature for 6 months. Prior to use, the plates are washed 2× with in each case 100 μl of PBS in order to wash away any possible unbound DNA.

### EXAMPLE 1e

Depleting Cells in Microtiter Plates —HYCOMP

Cells which are hybridized with polynucleotide probes (see item 1c) are washed 2× with PBS in order to remove excess probe and finally taken up in microtiter plate buffer (MP buffer: 5×SSC, 0.02% SDS, 2% blocking reagent (Roche), 0.1% N-laurylsarcosine, 33% formamide) and distributed between several microtiter wells which are coated with DNA which is complementary to the RNA probe (see item 1). The depletion takes place in a volume of 50 μl. Up to 1 μl of cell suspension (based on the quantity of cells employed during the hybridization with the probes [see item 1c]) is used per well. Accordingly, when the initial quantity employed is 10 μl of cell suspension, the cells are taken up in 350 μl of MP buffer and distributed between 10 microtiter wells including one well which serves as the negative control. The negative control consists of a microtiter well which is coated with a DNA which is different from that which is complementary to the probe.

The microtiter plates are incubated at 53° C. for from 1 to 2 h. In order to increase the depletion efficiency still further, it is possible, after that, to transfer the supernatant into fresh wells and incubate it at 53° C. for a further hour.

After this depletion step, the supernatant is carefully removed from the wells and can be used for further microscopic or molecular biological analysis. When the supernatant is being removed from the wells, care must be taken to ensure that the bottom of the wells is not contacted in order not to inadvertently remove cells which are bound to it.

### EXAMPLE 1f

Detection in the Microtiter Plates

The fact that the cells have been successfully bound to the plates can be determined with the aid of a streptavidin-peroxidase conjugate (when using biotin-labeled probes) or of an anti-DIG-peroxidase conjugate (in the case of DIG-labeled probes).

The wells are first of all washed 1× with 100 μl of PBS. After that, 100 μl of blocking buffer (PBS/1% blocking reagent (Roche)) are added and the wells are incubated at room temperature for 15 min. The supernatant is discarded and 50 μl of streptavidin-peroxidase solution (SA-POD, 100 mU per ml, diluted in blocking buffer) or anti-DIG-peroxidase solution (anti-DIG-POD, 150 mU per ml in blocking buffer) are added and the wells are incubated at room temperature for 30 min. The wells are then washed 3× with 100 μl of PBS. After the substrate BM-blue (Roche) has been added, a color reaction (change to blue) takes place, with this reaction being stopped after from 10 to 15 min by adding 100 μl of 1M H₂SO₄ (change to yellow). The color intensity can be measured in a photometer at a wavelength of 450 nm against a reference wavelength of 650 nm.

### EXAMPLE 1g

Detection in the Microscope

In order to check the specificity of the probe and the success of the depletion, the cells which are present in the supernatant in the wells after the depletion can be detected using fluorescent dyes (streptavidin-fluorescein when using biotin probes or anti-DIG-fluorescein in the case of DIG probes) and analyzed in the microscope. For this purpose, the cells are centrifuged down, taken up in 10 μl of H₂O, applied onto a microscope slide field and dried at 60° C. in an oven. The microscope slide field is then overlaid with 40 μl of a fluorescent dye solution (streptavidin-fluorescein (Roche, 5 μg per ml) diluted in DPBS, or anti-DIG-fluorescein (Roche, 40 μg/ml, in DPBS:137 mM NaCl, 2.7 mM KCl, 8 mM Na₂HPO₄) and incubated in the dark for 45 minutes. The solution is rinsed off with H₂O and the slide is washed in the dark for 15 min by immersing in DPBS, after which it is dried and covered with a cover slip. The analysis takes place in an epifluorescence microscope using an appropriate filter.

### EXAMPLE 2

Eukaryotes

On the one hand a mouse cell line (NIH-3T3 fibroblast) and, on the other hand, a human cell line (Jurkat cells, human T cells) were used as described in Example 1. Since, in contrast to bacteria and yeasts, mammalian cell lines do not possess any cell wall, but are only surrounded by a cell membrane, it was to be expected that the probes would be easily able to penetrate into the cell. Indeed, a strong hybridization signal was observed in both cell lines investigated, with this signal already appearing after from 2 to 3 hours (at the very earliest after approx. 5 h in the case of bacteria and yeasts). Different cell fixing methods (4% PFA, 100% EtOH, 4% PFA/70% EtOH) did not have any effect on the hybridization result. The clear signal obtained in the microscope shows that it is possible to immobilize the cells in the microtiter plates. However, it was not possible, with the rRNA-oriented probes which were used here, to effect any specific concentration/depletion from mixtures of the two cell lines investigated since humans and mice are virtually identical at the rRNA level and the probes are therefore not specific.

### EXAMPLE 3

Plasmids (High-Copy/Medium-Copy/Low-Copy)-beta-Lact Probe

Employing the method described in Example 1, plasmids were used instead of rRNA as anchor molecules for a polynucleotide probe. The three plasmids pCR2.1TOPO, copy number approx. 200, pBBR1MCS4, copy number approx. 50-100, and pUN121, copy number approx. 20-50, contain the beta-Lactamase gene as selection marker. An approx. 850 bp long segment from this sequence was used as the target region for the polynucleotide probe. In the case of all three plasmids, it was possible to observe a clear halo in the microscope and to successfully perform a depletion. At the same time, it was not possible to establish any significant variations in the signal intensity obtained with the high-copy/medium-copy and low-copy plasmids. Overall, however, the signals are weaker than in the case of the rRNA-oriented probes. From experiment to experiment, the depletion efficiency varies between 5 and 50%. It was found that longer hybridization times (from 18 to 24 hours) and a lower stringency (adjusted by way of the formamide content in the hybridization buffer; in this present case from 5 to 20%) are required in the case of plasmid-oriented probes as compared with probes which are directed against rRNA (comparison values for rRNA-oriented probes: from 5 to 16 h of hybridization; from 80 to 95% formamide in the hybridization buffer). The results are presented in the microscope photographs in FIGS. 2a, 2b and 2c.

### EXAMPLE 4

Genomic DNA—GAPDH Probe

A segment from gyceraldehyde-3-phosphate dehydroganse (GAPDH) was used as the target region. When employing the methods described in Example 1 and adjusting some reaction parameters, it was also possible, in this case, to observe a halo in the microscope and to successfully perform a depletion. These signals are weaker than those obtained with plasmid-oriented probes. The depletion efficiency was in the range from 5 to 35%. As compared with plasmid-oriented probes, the stringency had to be decreased still further (from 5 to 10% formamide) while the hybridization time had to be extended still further (from 24 to 30 h). Positive hybridization signals were observed in several independent experiments and the specificity of the probe was demonstrated using negative controls. FIG. 3 shows the result in the form of the microscope photographs which were obtained.

GAPDH probes were tested both on E. coli and on eukaryotes (NIH-3T3 and Jurkat cells). As expected, the signal was observed to be concentrated on the region of the cell nucleus in the case of the eukaryotic cells. As with the rRNA-oriented probes, a hybridization signal can also in this present case be observed substantially earlier in the eukaryotes (after from 4 to 5 h) than in the bacterial cells.

1. A method for sorting or for detecting at least one target cell, which contains at least one sought-after nucleotide sequence, in a cell mixture which contains this target cell,
characterized in that
(1) the cell mixture is treated, under hybridization conditions, with at least one polynucleotide as probe, which polynucleotide contains at least one segment whose sequence is complementary to the nucleotide sequence in the target cell,
(2) the cell mixture which has been treated in this way is contacted, under hybridization conditions, with a polynucleotide which is immobilized on a solid support and which is complementary to at least a segment in the probe polynucleotide, and
(3) the target cells which are immobilized on the solid support are separated from cells which are not immobilized.

2. The method as claimed in claim 1,
characterized in that
the polynucleotide probe is an RNA probe and/or DNA probe.

3. The method as claimed in claim 1 or 2,
characterized in that
use is made of a probe which is complementary to an rRNA, to a plasmid or to a DNA segment in a cell type in the cell mixture.

4. The method as claimed in one of the preceding claims,
characterized in that
use is made of a polynucleotide probe which has a length of at least 50 nucleotides.

5. The method as claimed in one of the preceding claims,
characterized in that
a microtiter plate which is coated with the complementary nucleotide sequence is used as the solid support for isolating non-immobilized cells from the cell mixture.

6. The method as claimed in one of claims 1 to 4,
characterized in that
a particulate material is used as the solid support which is coated with the complementary nucleotide sequence for isolating the cell type which can be immobilized on the support.

7. The method as claimed in one of the preceding claims,
characterized in that
a labeled polynucleotide probe is used.

8. The method as claimed in one of the preceding claims,
characterized in that
use is made of a polynucleotide probe which comprises sequence segments containing
(i) at least a first polynucleotide sequence which is complementary to a highly variable nucleotide sequence in the target cell, and
(ii) at least a second polynucleotide sequence which is complementary to a polynucleotide sequence which is immobilized on a solid support.
